(19) United States
(12) Patent Application Publication
(10) Pub. No.: US 2021/0137818 A1
(43) Pub. Date: May 13, 2021

(54) OIL-BASED HAIR COLORING COSMETIC

(71) Applicant: JO Cosmetics Co., Ltd., Tokyo (JP)

(72) Inventors: Taichi MORISHIMA, Tokyo (JP); Yusuke AKIZUKI, Tokyo (JP)

(73) Assignee: JO Cosmetics Co., Ltd., Tokyo (JP)

(21) Appl. No.: 16/476,626

(22) PCT Filed: Jan. 5, 2018
(86) PCT No.: PCT/JP2018/000059

§ 371 Date: Jul. 9, 2019

(30) Foreign Application Priority Data
Jan. 11, 2017 (JP) 2017-002577

(51) Int. Cl. A61K 8/891; A61K 8/02; A61K 8/86; A61K 8/92; A61K 8/60; A61K 8/37; A61K 8/67; A61K 8/19; A61K 8/25; A61K 8/34; A61Q 5/06; A61Q 1/10; A61K 8/73

(52) U.S. Cl. CPC A61K 8/891; A61K 2800/432; A61K 8/86; A61K 8/92; A61K 8/922; A61K 8/602; A61K 8/375; A61K 8/678; A61K 8/19; A61K 8/25; A61K 8/347; A61K 8/342; A61Q 5/065; A61Q 1/10; A61K 8/732; A61K 2800/48; A61K 8/0229

(57) ABSTRACT

An oil-based hair coloring cosmetic which contains 3 to 40% by mass of a solid oil having a melting point of 50° C. to 120° C. (A-1) and/or a lipophilic gelling agent (A-2), 1 to 15% by mass of an oil-soluble resin (B), 5 to 60% by mass of a volatile oil (C), 0.1 to 10% by mass of a nonionic surfactant having an HLB value of 5 to 11 (D), 3 to 70% by mass of a powder consisting of a coloring pigment and an extender pigment optionally used in combination with the coloring pigment (E), and 0 to 10% by mass of a non-volatile oil (F), wherein a ratio by mass of the component (F) to the component (B)[(F)/(B)] is 1 or less. This oil-based hair coloring agent has excellent water resistance and color transfer resistance, while being easily removed by shampooing.

### DETAILED DESCRIPTION OF THE INVENTION

### Technological Field

The present invention relates to an oil-based hair coloring cosmetic used as a temporary hair dye. More specifically, the invention relates to an oil-based hair coloring cosmetic which is excellent in water resistance, color transfer resistance and removability by shampooing.

### BACKGROUND TECHNOLOGY

Hair coloring cosmetics are generally classified into (1) permanent hair dye in the case of which an oxidative dye is used to produce a color in hair (sometimes referred to as a hair dye, hair coloring or the like), (2) semi-permanent hair dye in the case of which a dye such as an acid dye colors hair by its penetration and adsorption thereto (sometimes referred to as a hair manicure, color treatment or the like), and (3) temporary hair dye in the case of which hair is colored by forming a colored film on hair which includes a coloring material containing a pigment as a main component. This temporary hair dye is also referred to as hair coloring, and is referred to as hair mascara, hair color stick, and the like in accordance with the dosage form. Of these, the temporary hair dye is preferred by consumers as a hair coloring cosmetic that can be freely enjoyed because it has less damage to the hair, good removability by shampooing, and convenience of use.

On the other hand, the temporary hair dye (hair coloring cosmetic) has disadvantages that color retention and water resistance are generally insufficient as compared with the permanent hair dye, and color transfer tends to occur (that is, secondary adhesion is likely to occur). Various studies have conventionally been conducted to improve such disadvantages of the temporary hair dye. For example, Patent Document 1 discloses that a combination of a volatile oil, a water-repellent polymer, a powder, and non-volatile oil provides a hair dye which does not cause secondary adhesion after coating, and is excellent in color retention. Most of hair dyes specifically shown in the examples of Patent Document 1 contain a large amount of a silicone resin used as a water-repellent polymer, or a large amount of a nonvolatile oil. The hair dyes containing these components in a large amount are excellent in terms of improvement in water resistance and suppression of secondary adhesion, while they have a disadvantage that it is difficult to wash them off by shampooing.

In Patent Document 2, hair coloring cosmetics which contain a solid oil having a melting point of 55° C. or more, isostearic acid, and inorganic coloring pigments are proposed. The document discloses that the hair coloring cosmetics have no stiffness, and are excellent in durability and removability by shampooing. Hair dyes specifically shown in the examples of Patent Document 2 contain a nonvolatile oil such as isostearic acid and liquid paraffin in a large amount, while containing little resin and volatile oil. According to the experiments of the present inventors, it is revealed that the hair coloring cosmetic having such a formulation does not necessarily exhibit sufficient properties in water resistance and color transfer resistance.

### PRIOR ART DOCUMENT

### Patent Document

Patent Document 1: Japanese Unexamined Patent Publication No. H10-265354

### Patent Document 2: Japanese Unexamined Patent Publication No. 2014-97974

### DISCLOSURE OF THE INVENTION

### Problems to be Solved by the Invention

The present invention has been completed under such a background art. The present invention aims to provide an oil-based hair coloring cosmetic which is excellent in water resistance and color transfer resistance, and capable of being easily removed by shampooing, while maintaining convenience and little damage to hair which are advantages of conventional hair coloring cosmetics.

### Means Used to Solve the Problem

The present inventors have intensively studied to solve the above problems, and have found that a combination of use of a solid oil, an oil-soluble resin, a volatile oil and a powder in combination, use of a nonionic surfactant having an HLB within a specific range and use of a nonvolatile oil within a limited amount leads to an oil-based hair coloring cosmetic which is excellent in water resistance and color transfer resistance, and can be easily removed by shampooing. The present invention was completed based on such knowledge.

Thus, the present invention provides an oil-based hair coloring cosmetic comprising: (A) 3 to 40% by mass of a solid oil having a melting point of 50° C. to 120° C. and/or a lipophilic gelling agent, (B) 1 to 15% by mass of an oil-soluble resin, (C) 5 to 60% by mass of a volatile oil, (D) 0.1 to 10% by mass of a nonionic surfactant having an HLB value of 5 to 11, (E) 3 to 70% by mass of a powder consisting of a coloring pigment and an extender pigment optionally used in combination with the coloring pigment, and (F) 0 to 10% by mass of a nonvolatile oil, wherein a ratio by mass of the component (F) to the component (B), which is represented by (F)/(B), is 1 or less.

### Effect of the Invention

The oil-based hair coloring cosmetic of the present invention is excellent in hair dyeing effect and convenient usability because it is excellent in water resistance and color transfer resistance, and can be easily removed by shampooing.

### EMBODIMENTS FOR CARRYING OUT THE INVENTION

The oil-based hair coloring cosmetic of the present invention contains, as an essential component, a solid oil component and/or lipophilic gelling agent (A), an oil-soluble resin (B), a volatile oil (C), an nonionic surfactant having an HLB value of 5 to 11 (D), and a powder consisting of a coloring pigment and an extender pigment optionally used in combination with the coloring pigment (E), and further may contain a non-volatile oil (F) as a preferred optional component.

The component (A) is selected from a solid oil having a melting point of 50 to 120° C. (A-1), an oily gelling agent (A-2), and a mixture thereof. Such component (A) contributes to adhesion of the component (E) (i.e., powder) to hair along with the component (B) (i.e., oil-soluble resin) mentioned later, and imparts a viscosity or a hardness to the resulting oil-based coloring hair cosmetic. In the case that the cosmetic has a liquid form, the component (A) contributes to a stability of the cosmetic due to preventing sedimentation of a powder, and in the case that the cosmetic has a solid form, the component (A) contributes to stably holding a shape such as a stick or a cake.

(A-1: Solid Oil)

In the present invention, the component (A-1) is an oil which is solid at room temperature, and has a melting point of 50 to 120° C., preferably 55 to 105° C., more preferably 60 to 100° C. The melting point of the solid oil is a value measured according to a second method of the melting point measurement method which is a general test method defined in Japanese Standards of Quasi-drug Ingredients. When the component (A-1) has an excessively low melting point, water resistance and shape retention property become insufficient, while when it has an excessively high melting point, there is a disadvantage in production process of the cosmetic that operation at a high temperature is required upon melting along with the component (C) (i.e., volatile oil).

Examples of the solid oil includes hydrocarbon waxes (e.g., paraffin wax, polyethylene wax, ethylene propylene copolymer, microcrystalline wax, ceresin, ozokerite, synthetic wax, Fischer-Tropsch wax), Japan wax, carnauba wax, candelilla wax, rice wax, beeswax, hydrogenated jojoba oil, hydrogenated oil, higher alcohol, silicone wax, and the like.

Examples of the solid oil which is commercially available include paraffin waxes such as PARAFFIN WAX 135, PARAFFIN WAX 140, PARAFFIN WAX 150, and HNP-11 any of which is available from Nippon Seiro Co. Ltd.; microcrystalline waxes such as HNP-9, HI-MIC-2065, HI-MIC-1070, HI-MIC-1080, HI-MIC-1090, and HNP-0190 any of which is available from Nippon Seiro Co. Ltd., and MULTIWAX W-445 available from Sonneborn, LLC; polyethylene waxes such as PERFORMALENE 400, PERFORMALENE 500, and PERFORMALENE 655 any of which is available from NEW PHASE TECHNOLOGIES Inc.; synthetic waxes such as CIREBELLE 108 and CIREBELLE 305 both of which are available from CIREBELLE Inc.; candelilla waxes such as PURIFIED CANDELILLA WAX NO. 1, CANDELILLA NC 1630 both of which are available from Cerarica NODA Co., Ltd., PURIFIED CANDELILLA WAX CG-7, PURIFIED CANDELILLA WAX SR-3, HIGH MELTING POINT CANDELILLA WAX FR100 any of which available from Japan Natural Products Co., Ltd.

(A-2: Lipophilic Gelling Agent)

In the present invention, the component (A-2) is a lipophilic gelling agent which can be dissolved or dispersed in an oil being liquid at room temperature, and can act to thicken or gel the oil at the temperature. Examples of the lipophilic gelling agent include dextrin fatty acid esters, inulin fatty acid esters, sucrose fatty acid esters, starch fatty acid esters, organic modified clay minerals, metal soap, 12-hydroxystearic acid, and the like.

The dextrin fatty acid ester is an ester of dextrin or reduced dextrin and a higher fatty acid, and examples thereof include dextrin palmitate and dextrin Palmitate/2-ethylhexanoate. Examples of commercially available products include dextrin palmitate such as RHEOPEARL KL2 and RHEOPEARL TL2; dextrin palmitate/2-ethylhexanoate such as RHEOPEARL TT2, any of which is available from Chiba Flour Milling Co., Ltd.

The inulin fatty acid ester is an ester of inulin, which is a kind of fructooligosaccharides, and a higher fatty acid. Preferable inulin to be used has an average molecular weight of 300 to 10,000. Examples of the inulin fatty acid ester include inulin stearate, and examples of commercially available products include RHEOPEARL ISK2 manufactured by Chiba Flour Milling Co., Ltd.

The sucrose fatty acid ester is oil-soluble, and examples thereof include sucrose stearate and sucrose acetate stearate. Examples of commercially available products include SUGAR WAX S-10E, COSMELIKE S-10, and SUGAR WAX A-10E, any of which is available from DKS Co. Ltd.

Examples of the organically modified clay mineral include those obtained by substituting an exchangeable cation interposed between crystal layers of a clay mineral such as montmorillonite, saponite, hectorite and bentonite with an organic polar compound or an organic cation. Specific examples include dimethyl distearyl ammonium hectorite (i.e. di steardimonium hectorite), dimethyl distearyl ammonium bentonite (i.e. quaternium-18 bentonite), dioctadecyl dimethyl ammonium salt-modified montmorillonite, octadecyl dimethyl benzyl ammonium salt-modified montmorillonite, dihexadecyldimethyl ammonium salt-modified montmorillonite. Examples of commercially available products include Bentone 38V (disteardimonium hectorite), Bentone 34 (quaternium-18 bentonite), Bentone 27 (benzyl dimethyl distearyl ammonium hectorite), any of which is available from Elementis Corporation. Examples of the metal soap include aluminum stearate and calcium stearate.

The component (A) can be used alone or in combination of two or more. The content of the component (A) is 3 to 40% by mass, preferably 6 to 30% by mass, more preferably 7 to 25% by mass relative to the whole composition. When the content of the component (A) is too small, water resistant becomes insufficient and stability of the composition is lowered since powders tend to readily settle. While, when the content of component (A) is too large, removability by shampooing decreases, and also applicability to hair becomes lowered due to poor elongation upon use. In the case of preparing a solid cosmetic, (A-1) a solid oil having a melting point of 50 to 120° C. is preferably used as the component (A), thereby good shape retention can be obtained. In the case of preparing a liquid or pasty cosmetic, (A-2) a lipophilic gelling agent is preferably used as the component (A).

(B: Oil-Soluble Resin)

The oil-soluble resin used as component (B) is a compound which is soluble in oily components contained in a cosmetic, and acts to form a film on hair after volatilization of a volatile oil component. Incorporation of the component (B) makes the component (E) (i.e., powder) readily adhere to hair. The oil-soluble resin is not particularly limited as long as it is generally used for preparing cosmetics. Examples of the oil-soluble resin include silicone resin such as trimethylsiloxysilicate, partially crosslinked organopolysiloxane, tri(trimethylsiloxy)silylpropylcarbamoyl pullulan, polymethylsilsesquioxane, polypropylsilsesquioxane, fluorine modified silicone, acrylic modified silicone and silicone dendrimer modified resin compound; rosin acid-derived resin such as hydrogenated pentaerythrityl rosinate and hydrogenated glyceryl abietate; Candelilla resin; polyvinyl acetate type resin; polyvinyl isobutyl ether; polyisobutylene; and the like. Here, Candelilla resin means a resin component obtained by separating and extracting Candelilla wax with an organic solvent, and has a resin content of preferably 65% or more by mass, more preferably 85% or more by mass.

Of these, the silicone resin is excellent in water resistance and color transfer resistance. In particular, trimethylsiloxysilicate is preferably used. Examples of commercially available oil-soluble resin include X-21-5595, KF-7312J, and KF-7312F any of which is a solution of trimethylsiloxysilicate, and is available from Shin-Etsu Chemical Co., Ltd., FA4001CM SILICONE ACRYLATE available from Toray Corning Co., Ltd. which is a solution of a silicone dendrimer modified resin compound, 670FLUID available from Toray Corning Co., Ltd. which is a solution of polypropylsilsesquioxane, PINECRYSTAL KE-311 available from Harima Kasei Co. Ltd. which is a hydrogenated glyceryl rosinate, CANDELILLA RESIN E-1 available from Japan Natural Products Inc., and the like. These may be used alone or in combination as appropriate.

The content of the component (B) is 1 to 15% by mass, preferably 3 to 13% by mass, more preferably 5 to 12% by mass % relative to the whole composition. When the content is too small, water resistance and color transfer resistance become insufficient. When the content is too large, the hair coloring cosmetic is hardly removed by shampooing.

(C: Volatile Oil)

The volatile oil used as the component (C) is not limited as long as it is used in conventional cosmetics. The volatile oil usually has a boiling point at normal pressure of 60° C. to 260° C., preferably 100° C. to 220° C. Examples thereof include cyclic silicone oils such as octamethylcyclotetrasiloxane, decamethylcyclopentasiloxane and dodecamethylcyclohexasiloxane; low molecular weight silicone oils such as dimethicone and methyltrimethicone; hydrocarbon oils such as isododecane, isohexadecane and saturated isoparaffin-type hydrocarbon oils containing a compound having 8 to 16 carbon atoms as a main component (e.g. hydrogenated polyisobutene).

Examples of commercially available products include KF-994 (octamethylcyclotetrasiloxane), KF-995 (decamethylcyclopentasiloxane), KF-96A-lcs (octamethyltrisiloxane), KF-96L-1.5Cs (decamethyltetrasiloxane), and TMF-1.5 (methyl trimethicone) any of which is available from Shin-Etsu Chemical Co., Ltd.; MARCASOL R (isododecane) available from Maruzen Petrochemical Co., Ltd.; IP SOLVENT 1620 and IP SOLVENT 2028 available from Idemitsu Kosan Co., Ltd. any of which is a hydrogenated polyisobutene. Of these, in view of storage stability and volatilization rate, octamethyltrisiloxane, methyl trimethicone, isododecane and hydrogenated polyisobutene containing a compound having 8 to 16 carbon atoms as a main component are preferably used.

The content of the component (C) used is 5 to 60% by mass, preferably 10 to 55% by mass, more preferably 15 to 50% by mass relative to the whole composition. When the content is too small, it becomes difficult to uniformly apply the cosmetic to a desired portion of hair due to poor spreadability thereof. When the content is too large, coloring effect decreases due to a reduced amount of residual components on hair.

(D: Surfactant)

In the present invention, a nonionic surfactant having an HLB value of 5 to 11 is used as the component (D). When the nonionic surfactant has an HLB value of less than 5, removability by shampooing becomes insufficient. Conversely, when the nonionic surfactant has an HLB value of more than 11, water resistance becomes lowered. In particular, when the nonionic surfactant has an HLB value of 6 to 10, good balance of water resistance and removability by shampooing is obtained.

The component (D) is preferably a nonionic surfactant having an HLB value of 6 to 8 and being a liquid at 25° C. or a nonionic surfactant having an HLB value of 6 to 10 being a solid at 25° C. In view of water resistance and color transfer resistance, the solid nonionic surfactant is preferably used in particular. The component (D) may be a single nonionic surfactant, or may also be a combination of plural nonionic surfactants. When the component (D) is a combination of the liquid nonionic surfactant with the solid nonionic surfactant, a content of the solid nonionic surfactant is preferably 50% by mass or more, further preferably 70% by mass or more relative to the whole component (D) in view of water resistance and color transfer resistance. In the present invention, it is important to use the nonionic surfactant having an HLB value within the above-mentioned range. Even if an anionic surfactant, a cationic surfactants or an amphoteric surfactant is used in place of the nonionic surfactant, an oil-based hair coloring cosmetic which is excellent in water resistance, color transfer resistance and removability by shampooing cannot be obtained.

Incidentally, the term “HLB” is an index indicating a balance between hydrophilicity and lipophilicity as a value of from 0 to 20. The higher the lipophilicity, the closer to 0, the higher the hydrophilicity, the closer to 20. Regarding a method of determining an HLB value, various methods have conventionally been known. Further, an HLB value is described in catalogs provided by a manufacturer of nonionic surfactant. In the case of using a commercial nonionic surfactant, its HLB value indicates a value described in a catalog of a manufacturer. In the case of using a nonionic surfactant which is not a commercial product, its HLB value is determined in accordance with Method of Griffin which is described on page 307 of “Handbook of surfactants” published in 1960 from Sangyo Tosho publishing. The method is represented by the following formula.

HLB value=20×total weight of hydrophilic portion/molecular weight

Examples of the nonionic surfactant having an HLB value of 5 to 11 used as the component (D) include polyglycerol fatty acid esters such as polyglyceryl-4 stearate, polyglyceryl-2 oleate, polyglyceryl-10 distearate; polyoxyethylene hydrogenated castor oil such as PEG-10 hydrogenated castor oil and PEG-20 hydrogenated castor oil; polyoxyethylene fatty acid esters such as PEG-2 stearate and PEG-5 stearate; polyoxyethylene alkyl ethers such as ceteth-2, oleth-3 and steareth-6; fatty acid polyoxyethylene alkyl ethers such as steareth-6 stearate, laureth-8 isostearate and steareth-12 stearate; polyoxyethylene fatty acid glyceryl such as PEG-3 glyceryl isostearate, PEG-15 glyceryl triisostearate, PEG-10 glyceryl tristearate and PEG-20 glyceryl tristearate; fatty acid polyoxyethylene hydrogenated castor oil such as PEG-15 hydrogenated castor oil isostearate and PEG-20 hydrogenated castor oil triisostearate; polyoxyethylene sorbitan fatty acid ester, polyoxyethylene polyoxypropylene copolymer, ether of polyoxyethylene polyoxypropylene copolymer and long-chain alcohol, ether of polybutylene glycol polyglycerin copolymer and long-chain alcohol, and the like. Of these, polyoxyethylene fatty acid ester, polyoxyethylene fatty acid glyceryl, fatty acid polyoxyethylene hydrogenated castor oil and fatty acid polyoxyethylene alkyl ether are preferably used in view of water resistance and removability by shampooing.

When the nonionic surfactant used as the component (D) contains a fatty acid residue in its molecule, it is preferably a residue of a fatty acid having 10 to 22 carbon atoms such as myristic acid, palmitic acid, stearic acid, isostearic acid and oleic acid.

Examples of the liquid nonionic surfactant which is a commercial product include EMALEX RWIS-320 (PEG-20 hydrogenated castor oil triisostearate; Nihon Emulsion Co., Ltd.; HLB 6), EMALEX GWS-315 (PEG-15 glyceryl tristearate; Nihon Emulsion Co., Ltd.; HLB 7), and UNIOX GT-201S (PEG-20 glyceryl triisostearate; NOF Corporation; HLB 8). Examples of the solid nonionic surfactant commercially available include EMALEX GWS-320 (PEG-20 glyceryl tristearate; Nihon Emulsion Co., Ltd.; HLB 8), EMALEX SWS-12 (steareth-12 stearate; Nihon Emulsion Co., Ltd.; HLB 8), and EMALEX 608 (steareth-8; Nihon Emulsion Co., Ltd.; HLB 9).

The content of the component (D) used is 0.1 to 10% by mass, preferably 0.5 to 8% by mass, more preferably 1 to 6% by mass and further more preferably 1.5 to 4% by mass relative to the whole composition. When the content is too small, removability by shampooing decreases, whereas when it is too large, water resistance decreases.

In the present invention, in addition to the component (D), a hydrophilic surfactant selected from a nonionic surfactant having an HLB value of more than 11, an anionic surfactant, a cationic surfactant and an amphoteric surfactant can be appropriately used in combination as long as the effects of the present invention are not substantially impaired. Since these surfactants tend to lower water resistance of the cosmetic, in the case of containing these surfactants, it is appropriate to limit the content thereof to 2% by mass or less, more preferably 0.5% by mass or less, further more preferably 0.1% by mass or less relative to the whole composition. The content of these hydrophilic surfactants is preferably 10% by mass or less, more preferably 5% by mass or less relative to the total mass of the component (D). On the other hand, a nonionic surfactant having an HLB value of less than 5 may be contained as appropriate since it functions as an oil component. But, its content is preferably 10% by mass or less, more preferably 5% by mass or less relative to the whole composition since color transfer resistance decreases when it is excessively large.

(E: Powder)

The powder used as the component (E) in the present invention is a coloring pigment to color hair or a mixture of the coloring pigment and an extender pigment which is optionally used. The powder is not limited as long as it is commonly used for preparing conventional cosmetics, and can be used regardless of shape such as a spherical shape, a needle shape and a plate shape; a particle size such as an aerosol, a fine particle and a pigment grade; and a particle structure such as a porous particle and a non-porous particle. The coloring pigment may be an inorganic pigment or an organic pigment. In view of removability by shampooing, the inorganic coloring pigment is preferable.

Examples of the inorganic coloring pigment include inorganic white pigments such as titanium oxide and zinc oxide; inorganic red pigments such as iron oxide, iron hydroxide and iron titanate; inorganic brown pigments such as γ-iron oxide; inorganic yellow pigments such as yellow iron oxide and ocher; inorganic black pigments such as black iron oxide and carbon black; inorganic violet pigments such as manganese violet and cobalt violet; inorganic green pigments such as chromium hydroxide, chromium oxide, cobalt oxide and cobalt titanate; inorganic blue pigments such as iron blue and ultramarine blue; glittering pigments such as titanium oxide-coated mica and titanium oxide-coated glass flake; and the like.

Examples of the organic coloring pigment include organic pigments such as Red No. 202, Red No. 226, Blue No. 404 and Yellow No. 401; lakes derived from a water-soluble dye such as Red No. 104, Yellow No. 4, Yellow No. 5 and Blue No. 1; and lakes derived from a natural dye such as carminic acid, laccaic acid and carthamine, and the like. Water-soluble dyes which do not yet form a lake can also be formulated. In the case of formulating the water-soluble dye, it is preferred to make its amount small or to use it in a form of complex with other powders so that its elution amount reduces since removability by shampooing tends to decrease due to dyeing of hair by the water-soluble dye.

The extender pigment is suitably used to adjust coloring power, hardness or easiness of application of the oil-based hair coloring cosmetic. Examples of the extender pigment include inorganic extender pigments such as talc, muscovite, synthetic mica, phlogopite, synthetic fluorophlogopite, sericite, zeolite, kaolin, bentonite, clay, silicic acid, silicic anhydride (silica), magnesium silicate, magnesium aluminum silicate, calcium silicate, barium sulfate, magnesium carbonate, boron nitride, bismuth oxychloride, alumina, zirconium oxide, and hydroxyapatite; organic extender pigments such as silicone powder, silicone elastic powder, polyurethane powder, cellulose powder, nylon powder, silk powder, PMMA powder, starch, polyethylene powder, lauroyllysine, and metal soap; and complexes of the inorganic extender pigment and the organic extender pigment. Of these, porous spherical silica, sericite, talc, and lauroyllysine that have a volume average particle diameter of 0.1 to 20 μm are preferably used in view of easiness of application to hair.

The powder used as the component (E) may be a surface-treated pigment. Surface treatment can be conducted in accordance with conventional methods. Examples of the method include baking treatment with silicones such as methyl hydrogen polysiloxane and (dimethicone/methicone) copolymer; treatment with fatty acid such as stearic acid; treatment with fatty acid metal soap such as aluminum stearate and zinc stearate; treatment with acylated amino acid; treatment with lipoamino acid which is a combination of treatment with acylated amino acid or its salt and treatment with fatty acid or its salt; treatment with fluorinated compound such as perfluoroalkyl phosphate; treatment with silylation agent; treatment with acidic ester compound such as isostearyl sebacate; and the like. Powders treated with a hydrophobizing surface treatment agent improve water resistance, while they tend to decrease removability by shampooing. Therefore, it is preferred to use a powder without surface treatment.

The content of the component (E) is 3 to 70% by mass, preferably 5 to 65% by mass, more preferably 10 to 55% by mass relative to the whole composition. The content of the coloring pigment contained in the component (E) is preferably 3 to 50% by mass, more preferably 5 to 45% by mass, further more preferably 10 to 40% by mass % relative to the whole composition. When the component (E) is contained within the above-mentioned range, it is possible to obtain a suitable coloring effect. When the content of the component (E) is too small, coloring effect becomes insufficient, and color transfer resistance decreases. While, when it is too large, it becomes difficult to spread the composition on hair. In the case of using a mixture of the coloring pigment and the extender pigment as the component (E), the content of the extender pigment is 5 to 90% by mass, preferably 10 to 80% by mass, more preferably 20 to 70% by mass relative to the total mass of the component (E). By formulating the extender pigment in such a ratio, stickiness due to an oil component can be suppressed, and color transfer resistance can be further improved.

(F: Nonvolatile Oil)

The oil-based hair coloring cosmetic of the present invention may contain a nonvolatile oil in a range of 10% by mass or less as an optional component (F). The term “nonvolatile oil” means an oil being a liquid at normal temperature and normal pressure, or a semisolid oil having a melting point of less than 50° C., and contains neither a volatile oil having a boiling point of 260° C. or less, nor a water-soluble component. A nonionic surfactant having an HLB value of less than 5 is included in the “nonvolatile oil” as long as the above-mentioned conditions are satisfied.

The content of the component (F), i.e. nonvolatile oil, is required to be 10% by mass or less relative to the whole composition since excessive addition of the nonvolatile oil lowers color transfer resistance. The content is preferably 8% by mass or less, more preferably 5% by mass or less. While, the addition of the nonvolatile oil improves an operability upon dissolving the component (A), i.e., solid oil, in the component (C), i.e., volatile oil, in the step of producing a hair coloring cosmetic, and also improves removability by shampooing. Hence, the content of the component (F) is 0.1% by mass or more, more preferably 0.5% by mass or more, further more preferably 1% by mass or more relative to the total composition.

The nonvolatile oil used as the component (F) functions as an adhesion agent to the component (E), i.e., pigment powder along with the component (B), i.e., oil-soluble resin. When the amount of component (F) is larger than that of the component (B), color transfer resistance is lowered. Hence, the ratio of the component (F) to the component (B) represented by the following formula is required to be 1 or less, preferably 0.5 or less, more preferably 0.4 or less.

Ratio by mass=Amount of Nonvolatile oil (F)/Amount of Oil-soluble resin (B)

The nonvolatile oil used in the present invention is not particularly limited as long as it is used in conventional cosmetics, and may be any animal oil, vegetable oil or synthetic oil. Examples of the nonvolatile oil include esters such as triethylhexanoin, diisostearyl malate, polyglyceryl-2 isostearate, diglyceryl triisostearate, decaglyceryl decaisostearate, oligomeric esters of dimer acid and dimer diol, pentaerythrityl tetraisostearate, diglyceryl tetraisostearate, cetyl isooctanoate, isopropyl myristate, isopropyl palmitate, octyldodecyl myristate, neopentyl glycol dioctanoate, cholesterol fatty acid ester and jojoba oil; hydrocarbons such as polybutene, polyisobutylene, heavy liquid isoparaffin, liquid paraffin, α-olefin oligomer, squalaene and petrolatum; fats and oils such as olive oil, castor oil, mink oil and macadamia nut oil; fatty acids such as isostearic acid and oleic acid; higher alcohols such as oleyl alcohol and isostearyl alcohol; silicone oils such as dimethyl polysiloxane having a low polymerization degree, dimethyl polysiloxane having a high polymerization degree, methylphenyl polysiloxane, cross-linked organopolysiloxane and fluorine-modified polysiloxane; fluorine type oils such as perfluoropolyether; lanolin derivatives such as lanolin, lanolin acetate, lanolin fatty acid isopropyl and lanolin alcohol; and the like. The nonvolatile oil of the component (F) may be a nonionic surfactant having an HLB value of less than 5 which is a liquid or pasty oil at room temperature.

The oil-based hair coloring cosmetic may contain a small amount of water unless the effects of the present invention are substantially impaired. The content of water is preferably 5% by mass or less with relative to the whole composition in view of formulation stability.

The oil-based hair coloring cosmetic may contain other components which are used in conventional cosmetics, for example, additives such as polyhydric alcohols, lower alcohols, ultraviolet absorbers, ultraviolet scattering agents, humectants, fragrances, antioxidants, preservatives, defoaming agents, fibers, and various extracts unless the effects of the present invention are substantially impaired.

The oil-based hair coloring cosmetic of the present invention can be prepared in accordance with conventional methods. For example, solid oil-based hair coloring cosmetics can be prepared by heating entire raw materials to a temperature higher than melting points thereof and homogeneously mixing them, then providing the mixture in a molten state to a container or mold followed by cooling or allowing to cool. Liquid or pasty oil-based hair coloring cosmetics can be prepared by heating entire raw materials to a temperature higher than melting points thereof and cooling the mixture while stirring. Also, the oil-based hair coloring cosmetic can be prepared by preliminarily mixing the coloring materials with a part of the oil component and/or the surfactant component to make a pre-mixture, subsequently combining the pre-mixture with the remaining ingredients, and heating the mixture.

The oil-based hair coloring cosmetic can be used as a hair color stick, a hair mascara, a hair concealer, a hair foundation, a gray hair hidden agent, an eyebrow coloring agent, and the like. Among them, it is particularly suitable as a gray hair hidden agent for applying to a specific part such as a hair line. The form of the cosmetic may be appropriately selected in accordance with its application. For example, the form can be liquid, pasty, solid or the like. The oil-based solid hair coloring cosmetic is used in the form of being filled in a suitable container such as metal pan, plastic pan and stick container. Hence, it has advantages that it is excellent in easiness of use and portability.

### EXAMPLES

Hereinafter, the present invention will be further described with reference to Examples and Comparative Examples, but the present invention is not limited by these Examples. The content of each ingredient in formulations described below is expressed in % by mass relative to the whole composition unless otherwise specified.

The evaluation methods of the oil-based hair coloring cosmetic in the following Examples and Comparative Examples are as follows.

### (Hair Bundle Used for Evaluation)

A hair bundle of human white hair of about 12 cm in length and about 0.9 g, which is available from Beaulax Corporation as BM-W-A, was washed with a commercial shampoo (Kao Corporation; Essential shampoo-cR), then was dried well. After applying 0.4 g of an oil-based hair coloring cosmetic for evaluation to the hair bundle and spreading it well with fingers, the hair bundle was air-dried for 20 minutes to obtain a colored hair bundle.

(Water Resistance)

The colored hair bundle was put into 500 mL of water in a glass beaker having an outer diameter of 90 mm and a height of 120 mm, and allowed to stand for 3 minutes. After taking out the bundle from the water, the bundle being in a wet state was sandwiched between a piece of filter paper having a diameter of 125 mm (Toyo Roshi Co., Ltd., qualitative filter paper, No. 1) which was folded in half. Then, a glass beaker having an outer diameter of 110 mm, a height of 150 mm and a total weight of 1.2 Kg which was adjusted by addition of water was placed on the filter paper so as to press the whole bundle, and allowed to stand for 1 minute. After taking out the bundle, the filter paper was dried for 24 hours in a thermostatic chamber at 25° C. As to each of the filter paper obtained as set forth above and the original filter paper, brightness was measured using a colorimeter (X-rite Co., SP60) to obtain brightness difference (ΔL*) between these filter papers. The measurement was conducted three times. Water resistance was evaluated in accordance with the following evaluation criteria. The criteria indicates that the larger the brightness difference (ΔL*) is, the more of the amount of the oil-based hair coloring cosmetic transfers to the filter paper from the colored bundle, that is, water resistance decreases.

(Evaluation Criteria)

5: ΔL* is less than 1

4: ΔL* is 1 or more and less than 5

3: ΔL* is 5 or more and less than 10

2: ΔL* is 10 or more and less than 15

1: ΔL* is 15 or more

(Color Transfer Resistance)

An oil-based hair coloring cosmetic for evaluation was applied to an artificial skin (trade name: BIOSKIN PLATE, Beaulax Corporation) in an amount of about 2 mg/cm², and allowed to stand for 20 minutes for drying. Then, a glass filter paper (Toyo Roshi Co., Ltd., GA100, diameter 45 mm) was placed on the artificial skin, subsequently a weight having a weight of 1 Kg and a diameter of 53 mm was placed on the glass filter paper. Then, the glass filter paper sandwiched between the artificial skin and the weight was pulled out with a constant force. Using a colorimeter (X-rite Co., SP60), brightness difference (ΔL*) between a portion of the glass filter paper where color transfer occurred and the original glass filter paper was measured. The brightness difference was indicated as an average value in measurements conducted three times. Color transfer resistance was evaluated in accordance with the following evaluation criteria. The criteria indicates that the larger the brightness difference (ΔL*) is, the more amount of the oil-based hair coloring cosmetic transfers to the glass filter paper from the colored bundle, that is, color transfer resistance decreases.

(Evaluation Criteria)

5: ΔL* is less than 3.5

4: ΔL* is 3.5 or more and less than 4

3: ΔL* is 4 or more and less than 4.5

2: ΔL* is 4.5 or more and less than 5

1: ΔL* is 5 or more

### (Removability by Shampooing)

An oil-based hair coloring cosmetic for evaluation was applied to human hair. After the elapse of 6 to 10 hours, the human hair was shampooed with a commercially available shampoo to evaluate removability by shampooing in accordance with the following evaluation criteria. Evaluation point is an average value of scores obtained by 10 evaluators.

(Evaluation Criteria)

5: Cosmetic was removed very well.

4: Cosmetic was removed well.

3: Inconspicuous but slight color remained.

2: Color remained.

1: Most color remained.

### (Easiness of Application)

Application property of an oil-based hair coloring cosmetic to human hair was evaluated in accordance with the following evaluation criteria in which ten evaluators respectively scored regarding easiness of application. Evaluation point is an average value of scores obtained by 10 evaluators.

### (Evaluation Criteria)

5: Very easy

4: Easy

3: Cannot say either

2: Difficult

1: Very difficult

### Examples 1 to 5 and Comparative Examples 1 to 2

### (Hair Color Stick)

A hair color stick of the formulation shown in Table 1 was prepared according to the following procedure, and was evaluated according to the above-mentioned methods as to water resistance, color transfer property, removability by shampooing, and easiness of application. The results are shown in Table 1.

### (Production Procedure)

(1) Ingredients contained in phase A shown in Table 1 were heated to about 110° C. and homogeneously mixed.
(2) Ingredients contained in phases B and C shown in Table 1 were added to the mixture prepared in the above (1), and the resultant mixture was homogeneously mixed at 90° C.
(3) The mixture prepared in the above (2) which was in a molten state was filled into a stick container at 80° C., and allowed to stand to form a hair color stick.

TABLE 1

| | | | | | | | | Comparatove | |
|---|---|---|---|---|---|---|---|---|---|
| | | | Example | | | | | Example | |
| Phase | Component | | 1 | 2 | 3 | 4 | 5 | 1 | 2 |
| A | A | Polyethylene wax (m.p. | 2.50 | 2.50 | 2.50 | 2.50 | 2.50 | 2.50 | 2.50 |
| | | 75-90° C.)( [symbol] 1) | | | | | | | |
| | A | Candelilla wax (m.p. | 1.00 | 1.00 | 1.00 | 1.00 | 1.00 | 1.00 | 1.00 |
| | | 70-75° C.)( [symbol] 2) | | | | | | | |
| | A | Synthetic wax (m.p. | 8.00 | 8.00 | 8.00 | 8.00 | 8.00 | 8.00 | 8.00 |
| | | 80-85° C.)( [symbol] 3) | | | | | | | |
| | A | Sucrose fatty acid ester | 0.50 | 0.50 | 0.50 | 0.50 | 0.50 | 0.50 | 0.50 |
| | | ( [symbol] 4) | | | | | | | |
| | F | Triethylhexanoin | 2.00 | 2.00 | 2.00 | 2.00 | 2.00 | 2.00 | 2.00 |
| | D | PEG-20 glyceryl tristearate | 2.00 | 1.00 | 3.00 | 5.00 | 8.00 | 0.00 | 12.00 |
| | | (HLB: 8)( [symbol] 5) | | | | | | | |
| | F | Sorbitan Sesquiisostearate | 0.50 | 0.50 | 0.50 | 0.50 | 0.50 | 0.50 | 0.50 |
| | | ( [symbol] 6) | | | | | | | |
| | F | Tocopherol | 0.05 | 0.05 | 0.05 | 0.05 | 0.05 | 0.05 | 0.05 |
| B | E | Black iron oxide | 11.00 | 11.00 | 11.00 | 11.00 | 11.00 | 11.00 | 11.00 |
| | E | Titanium oxide | 3.00 | 3.00 | 3.00 | 3.00 | 3.00 | 3.00 | 3.00 |
| | E | Red iron oxide | 6.00 | 6.00 | 6.00 | 6.00 | 6.00 | 6.00 | 6.00 |
| | E | Yellow iron oxide | 3.00 | 3.00 | 3.00 | 3.00 | 3.00 | 3.00 | 3.00 |
| | E | Sericite | 14.00 | 14.00 | 14.00 | 14.00 | 14.00 | 14.00 | 14.00 |
| | E | Silica ( [symbol] 7) | 3.00 | 3.00 | 3.00 | 3.00 | 3.00 | 3.00 | 3.00 |
| C | B/C | Trimethylsiloxysilicate/ | 20.00 | 20.00 | 20.00 | 20.00 | 20.00 | 20.00 | 20.00 |
| | | Decamethylpentasiloxane = | | | | | | | |
| | | 50/50 ( [symbol] 8) | | | | | | | |
| | C | Decamethylpentasiloxane | Balance | Balance | Balance | Balance | Balance | Balance | Balance |
| | | Phenoxyethanol | 0.50 | 0.50 | 0.50 | 0.50 | 0.50 | 0.50 | 0.50 |

| | | | | | | | | |
|---|---|---|---|---|---|---|---|---|
| Evalu- | Water resistance | 4 | 5 | 4 | 3 | 3 | 5 | 1 |
| ation | Color transfer resistance | 4 | 5 | 4 | 3 | 3 | 5 | 2 |
| | Romovability by shampooing | 5 | 3 | 5 | 5 | 5 | 1 | 5 |
| | Easiness of application | 4 | 4 | 4 | 4 | 4 | 4 | 4 |

( [symbol] 1)Product Name, PERFORMALENE 400 POLYETHYLENE (NEW PHASE TECHNOLOGIES Inc.)
( [symbol] 2)Product Name, CANDELILLA WAX NC1630 (Cerarica NODA Co., Ltd.)
( [symbol] 3)Product Name, CIREBELLE108 (DKSH, Inc.)
( [symbol] 4): Product Name, SUGAR WAX A-10E (Daiichi Kogyo Seiyaku Co., Ltd)
( [symbol] 5)Product name, EMALEX GWS-320 (Nihon Emulsion Co., Ltd.)
( [symbol] 6): Product name, NIKKOL SI-15RV (Nikko Chemicals Co., Ltd.)
( [symbol] 7): Product name, SILICA MICRO BEADS P-1500 (JGC catalysts & Chemicals Co., Ltd.)
( [symbol] 8): Product name, SILICONE KF-7312J (Shin-Etsu Chemical Co., Ltd.)

As is seen from the results shown in Table 1, the amount of nonionic surfactant used as the component (D) greatly affected water resistance, color transfer resistance, and removability by shampooing. That is, the hair color stick of Comparative Example 1 containing no PEG-20 glyceryl tristearate, which is a solid nonionic surfactant having an HLB value of 8, was excellent in water resistance and color transfer resistance, but was difficult to remove by shampooing, while hair color sticks of Examples 1 to 5 had remarkably improved removability by shampooing, and also had sufficient properties on water resistance, color transfer resistance and easiness of application. However, the hair color stick of Comparative Example 2 containing the nonionic surfactant in an amount of 12% by mass relative to the whole composition had no sufficient properties on water resistance and color transfer resistance.

### Examples 6 to 9 and Comparative Examples 3 to 5

### (Hair Color Stick)

Hair color sticks of the formulation shown in Table 2 were prepared according to the above-mentioned procedure, and were evaluated according to the above-mentioned methods as to water resistance, color transfer resistance, removability by shampooing, and easiness of application. The results are shown in Table 2.

TABLE 2

| | | | | | | | Comparative | | |
|---|---|---|---|---|---|---|---|---|---|
| | | | Example | | | | Example | | |
| Phase | Component | | 6 | 7 | 8 | 9 | 3 | 4 | 5 |
| A | A | Polyethylene wax ( [symbol] 1) | 2.50 | 2.50 | 2.50 | 2.50 | 2.50 | 2.50 | 2.50 |
| | A | Candelilla wax ( [symbol] 2) | 1.00 | 1.00 | 1.00 | 1.00 | 1.00 | 1.00 | 1.00 |
| | A | Synthetic wax ( [symbol] 3) | 8.00 | 8.00 | 8.00 | 8.00 | 8.00 | 8.00 | 8.00 |
| | A | Sucrose fatty acid ester ( [symbol] 4) | 0.50 | 0.50 | 0.50 | 0.50 | 0.50 | 0.50 | 0.50 |
| | F | Triethylhexanoin | 2.00 | 2.00 | 2.00 | 2.00 | 2.00 | 2.00 | 2.00 |
| | F | PEG-4 glyceryl tristearate (Solid, | — | — | — | | 2.00 | — | — |
| | | HLB: 2) ( [symbol] 9) | | | | | | | |
| | D | PEG-20 hydrogenated castor oil | 2.00 | — | — | | — | — | — |
| | | triisostearate (Liquid, HLB: 6)( [symbol] 10) | | | | | | | |
| | D | Steareth-12 stearate (Solid, HLB: 8) | — | 2.00 | — | | — | — | — |
| | | ( [symbol] 11) | | | | | | | |
| | D | PEG-20 glyceryl triisostearate | — | — | 2.00 | | — | — | — |
| | | (Liquid, HLB: 8)( [symbol] 12) | | | | | | | |
| | D | Steareth-8 (Solod, HLB: 8) ( [symbol] 13) | — | — | — | 2.00 | | | |
| | | Steareth-15 (Solid, HLB: 12) ( [symbol] 14) | — | — | — | | — | 2.00 | — |
| | | Polysorbate 80 (Liquid, HLB: 15) | — | — | — | | — | — | 2.00 |
| | | ( [symbol] 15) | | | | | | | |
| | F | Sorbitan Sesquiisostearate ( [symbol] 6) | 0.50 | 0.50 | 0.50 | 0.50 | 0.50 | 0.50 | 0.50 |
| | F | Tocopherol | 0.05 | 0.05 | 0.05 | 0.05 | 0.05 | 0.05 | 0.05 |
| B | E | Black iron oxide | 11.00 | 11.00 | 11.00 | 11.00 | 11.00 | 11.00 | 11.00 |
| | E | Titanium oxide | 3.00 | 3.00 | 3.00 | 3.00 | 3.00 | 3.00 | 3.00 |
| | E | Red iron oxide | 6.00 | 6.00 | 6.00 | 6.00 | 6.00 | 6.00 | 6.00 |
| | E | Yellow iron oxide | 3.00 | 3.00 | 3.00 | 3.00 | 3.00 | 3.00 | 3.00 |
| | E | Sericite | 14.00 | 14.00 | 14.00 | 14.00 | 14.00 | 14.00 | 14.00 |
| | E | Silica ( [symbol] 7) | 3.00 | 3.00 | 3.00 | 3.00 | 3.00 | 3.00 | 3.00 |
| C | B/C | Trimethylsiloxysilicate/ | 20.00 | 20.00 | 20.00 | 20.00 | 20.00 | 20.00 | 20.00 |
| | | Decamethylpentasiloxane = 50/50 | | | | | | | |
| | | ( [symbol] 8) | | | | | | | |
| | C | Decamethylpentasiloxane | Balance | Balance | Balance | Balance | Balance | Balance | Balance |
| | | Phenoxyethanol | 0.50 | 0.50 | 0.50 | 0.50 | 0.50 | 0.50 | 0.50 |

| | | | | | | | | |
|---|---|---|---|---|---|---|---|---|
| Evalu- | Water resistance | 5 | 4 | 3 | 4 | 5 | 1 | 1 |
| ation | Color transfer resistance | 5 | 4 | 4 | 4 | 5 | 4 | 4 |
| | Romovability by shampooing | 4 | 5 | 5 | 5 | 2 | 5 | 5 |
| | Easiness of application | 4 | 4 | 4 | 4 | 4 | 4 | 4 |

( [symbol] 9): Product name, EMALEX GWS-304 (Nihon Emulsion Co., Ltd.)
( [symbol] 10)Product name, EMALEX RWIS-320 (Nihon Emulsion Co., Ltd.)
( [symbol] 11): Product name, EMALEX SWS-12 (Nihon Emulsion Co., Ltd. Ltd.)
( [symbol] 12)Product name, UNIOX GT-20IS (NOF Corporation)
( [symbol] 13): Product name, EMALEX 608 (Nihon Emulsion Co., Ltd.)
( [symbol] 14): Product name, EMALEX 615 (Nihon Emulsion Co., Ltd.)
( [symbol] 15): Product name, RHEODOL TW-O120V (Kao Corporation)

As is seen from the results shown in Table 2, HLB value of the nonionic surfactant greatly affected water resistance and removability by shampooing. That is, the hair color stick of Comparative Example 3 using a solid and nonionic surfactant having an HLB value of 2 was excellent in water resistance and color transfer resistance, but was very insufficient in removability by shampooing. Further, both hair color sticks of Comparative Examples 4 to 5 which contain a solid and nonionic surfactant having an HLB value of 12 or a liquid and nonionic surfactant having an HLB value of 15 were very insufficient in removability by shampooing. In contrast, the hair color sticks of Examples 6 to 9 had remarkably improved removability by shampooing, and also had sufficient properties on water resistance, color transfer resistance and easiness of application. In particular, the hair color sticks of Examples 7 and 9 using a solid and nonionic surfactant were remarkably excellent in removability by shampooing as well as being excellent in water resistance and color transfer resistance.

### Example 10 and Comparative Examples 6 to 7

### (Hair Color Stick)

Hair color sticks of the formulation shown in Table 3 were prepared according to the above-mentioned procedure, and were evaluated according to the above-mentioned methods as to water resistance, color transfer resistance, removability by shampooing, and easiness of application. The results are shown in Table 3. For reference, the formulation and the results of Example 1 are described again in Table 3.

TABLE 3

| | | | | | Comparative | |
|---|---|---|---|---|---|---|
| | | | Example | | Example | |
| Phase | Component | | 1 | 10 | 6 | 7 |
| A | A | Polyethylene wax (m.p. 75-90° C.) ( [symbol] 1) | 2.50 | 2.50 | 2.50 | 2.50 |
| | A | Candelilla wax (m.p. 70-75° C.) ( [symbol] 2) | 1.00 | 1.00 | 1.00 | 1.00 |
| | A | Synthetic wax (m.p. 80-85° C.) ( [symbol] 3) | 8.00 | 8.00 | 8.00 | 8.00 |
| | A | Sucrose fatty acid ester ( [symbol] 4) | 0.50 | 0.50 | 0.50 | 0.50 |
| | F | Triethylhexanoin | 2.00 | 2.00 | 10.00 | 2.00 |
| | D | PEG-20 glyceryl tristearate (HLB: 8)( [symbol] 5) | 3.00 | 3.00 | 3.00 | 3.00 |
| | F | Sorbitan Sesquiisostearate( [symbol] 6) | 0.50 | 0.50 | 0.50 | 0.50 |
| | F | Tocopherol | 0.05 | 0.05 | 0.05 | 0.05 |
| B | E | Black iron oxide | 11.00 | 11.00 | 11.00 | 11.00 |
| | E | Titanium oxide | 3.00 | 3.00 | 3.00 | 3.00 |
| | E | Red iron oxide | 6.00 | 6.00 | 6.00 | 6.00 |
| | E | Yellow iron oxide | 3.00 | 3.00 | 3.00 | 3.00 |
| | E | Sericite | 14.00 | 14.00 | 14.00 | 14.00 |
| | E | Silica ( [symbol] 7) | 3.00 | 3.00 | 3.00 | 3.00 |
| C | B/C | Trimethylsiloxysilicate/ | 20.00 | 10.00 | 20.00 | 36.00 |
| | | Decamethylpentasiloxane = 50/50 ( [symbol] 8) | | | | |
| | C | Decamethylpentasiloxane | Balance | Balance | Balance | Balance |
| | | Phenoxyethanol | 0.50 | 0.50 | 0.50 | 0.50 |
| | | Total of Component (F) | 2.55 | 2.55 | 2.55 | 2.55 |
| | | Ratio of (F) to (B) | 0.26 | 0.51 | 1.06 | 0.14 |

| | | | | | |
|---|---|---|---|---|---|
| Evalu- | Water resistance | 4 | 3 | 2 | 5 |
| ation | Color transfer resistance | 5 | 3 | 1 | 5 |
| | Romovability by shampooing | 5 | 5 | 5 | 1 |
| | Easiness of application | 4 | 4 | 4 | 2 |

As shown in Table 3, the hair color stick of Example 10 which contains the component (B) in an amount of 5% by mass exhibited sufficient properties although water resistance and color transfer resistance were slightly lowered as compared with the hair color stick of Example 1 containing the component (B) in an amount of 10% by mass. On the other hand, when the content of the component (F) exceeded 10% by mass, water resistance and color transfer resistance significantly lowered (Comparative Example 6). Also, when the content of the component (B) exceeded 15% by mass, removability by shampooing and easiness of application were insufficient (Comparative Example 7).

### Example 11

### (Hair Mascara)

A hair mascara of the formulation shown in Table 4 was prepared according to the following procedure, and was evaluated according to the above-mentioned methods as to water resistance, color transfer resistance, removability by shampooing, and easiness of application. The result is shown in Table 4.

### (Production Procedure)

(1) Ingredients contained in phase A shown in Table 4 were heated to about 110° C. and homogeneously mixed.
(2) Ingredients contained in phases B and C shown in Table 4 were added to the mixture prepared in the above (1), and the resultant mixture was homogeneously mixed at 90° C.
(3) The mixture prepared in the above (2) was cooled to 35° C. while stirring, and filled into a container of mascara having a cap with a brush to form a hair mascara.

TABLE 4

| Phase | Component | | Example 11 |
|---|---|---|---|
| A | A | Dextrin palmitate ( [symbol] 16) | 5.00 |
| | A | Inulin stearate ( [symbol] 17) | 10.00 |
| | A | Sucrose fatty acid ester ( [symbol] 4) | 1.00 |
| | F | Triethylhexanoin | 3.00 |
| | C | Decamthylpentasiloxane | Balance |
| | D | PEG-20 glyceryl tristearate (HLB: 8)( [symbol] 5) | 2.50 |
| | F | Sorbitan Sesquiisostearate | 0.50 |
| | F | Tocopherol | 0.05 |
| B | E | Black iron oxide | 15.00 |
| | E | Sericite | 14.00 |
| | E | Silica ( [symbol] 7) | 3.00 |
| C | B/C | Trimethylsiloxysilicate/ | 20.00 |
| | | Decamethylpentasiloxane = 50/50 ( [symbol] 8) | |
| | | Phenoxyethanol | 0.50 |
| Evalu- | | Water resistance | 5 |
| ation | | Color transfer resistance | 4 |
| | | Romovability by shampooing | 5 |
| | | Easiness of application | 5 |

( [symbol] 16): Product name, RHEOPEARL KL2 (Chiba Flour Milling Co., Ltd.)
( [symbol] 17): Product name, RHEOPEARL ISK2 (Chiba Flour Milling Co., Ltd.)

As is apparent from the result shown in Table 4, the hair color mascara of Example 11 was excellent in water resistance, color transfer resistance, and removability by shampooing.

### INDUSTRIAL APPLICABILITY

According to the present invention, there is provided an oil-based hair coloring cosmetic which is excellent in removability by shampooing in addition to having excellent water resistance and color transfer resistance.

1. An oil-based hair coloring cosmetic comprising:
3 to 40% by mass of a solid oil having a melting point of 50° C. to 120° C. and/or a lipophilic gelling agent (A),
1 to 15% by mass of an oil-soluble resin (B),
5 to 60% by mass of a volatile oil (C),
0.1 to 10% by mass of a nonionic surfactant having an HLB value of 5 to 11 (D),
3 to 70% by mass of a powder consisting of a coloring pigment and an extender pigment optionally used in combination with the coloring pigment (E), and
0 to 10% by mass of a nonvolatile oil (F),
wherein a ratio by mass of the component (F) to the component (B), which is represented by (F)/(B), is 1 or less.

2. The oil-based hair coloring cosmetic according to claim 1, wherein the component (D) is a nonionic surfactant being solid at 25° C. and having an HLB value of 6 to 10, or a nonionic surfactants being liquid at 25° C. and having an HLB value of 6 to 8.

3. The oil-based hair coloring cosmetic according to claim 1, wherein the component (D) contains at least 50% by mass of the nonionic surfactant being solid at 25° C. and having an HLB value of 6 to 10.

4. The oil-based hair coloring cosmetic according to claim 1, wherein the component (F) is contained in an amount of 0.1 to 8% by mass relative to the total mass of the whole composition.

5. The oil-based hair coloring cosmetic according to claim 1, wherein the component (E) is a mixture of the coloring pigment and the extender pigment.

6. The oil-based hair coloring cosmetic according to claim 1, wherein the component (B) is a silicone resin.

7. The oil-based hair coloring cosmetic according to claim 1, wherein the component (D) is at least one selected from the group consisting of polyglycerol fatty acid ester, polyoxyethylene hydrogenated castor oil, polyoxyethylene fatty acid ester, polyoxyethylene alkyl ether, fatty acid polyoxyethylene alkyl ether, polyoxyethylene fatty acid glyceryl, fatty acid polyoxyethylene hydrogenated castor oil, polyoxyethylene sorbitan fatty acid ester, polyoxyethylene polyoxypropylene copolymer, polyoxyethylene polyoxypropylene etherified with a long-chain alcohol, and a polybutylene glycol polyglycerin copolymer etherified with a long-chain alcohol.

8. The oil-based hair coloring cosmetic according to claim 1, wherein when a hydrophilic surfactant selected from the group consisting of a nonionic surfactant having an HLB value of more than 11, an anionic surfactant, a cationic surfactant, and a hydrophilic surfactant is contained, a content of the hydrophilic surfactant is 2% by mass or less relative to the whole composition.

9. The oil-based hair coloring cosmetic according to claim 1, wherein the component (A) is the solid oil having a melting point of 50 to 120° C. (A-1), and the cosmetic has a solid form.

10. The oil-based hair coloring cosmetic according to claim 1, wherein the component (A) is the lipophilic gelling agent (A-2), and the cosmetic has a liquid or pasty form.
